(19) United States
(12) Patent Application Publication
(10) Pub. No.: US 2024/0062877 A1
(43) Pub. Date: Feb. 22, 2024

(54) SYSTEM AND METHOD FOR PROVIDING MENTAL AND BEHAVIOURAL HEALTH SERVICES

(71) Applicant: Sandeep VOHRA, New Delhi (IN)

(72) Inventor: Sandeep VOHRA, New Delhi (IN)

(21) Appl. No.: 18/386,513

(22) Filed: Nov. 2, 2023

Related U.S. Application Data
(63) Continuation of application No. PCT/IB23/53984, filed on Apr. 19, 2023.

(30) Foreign Application Priority Data
Apr. 22, 2022 (IN) 202211023699

(51) Int. Cl. G16H 20/70; G16H 10/20; G06V 40/16; G16H 10/60; G16H 80/00; G16H 50/30; G16H 50/20; G16H 15/00

(52) U.S. Cl. CPC G16H 20/70; G16H 10/20; G06V 40/174; G16H 10/60; G16H 80/00; G16H 50/30; G16H 50/20; G16H 15/00

(57) ABSTRACT

A system and a method for providing mental health or behavioural health services. The system includes a data generation module and a natural language processing module, which work together to capture and store user data related to their medical treatment. The data generation module is responsible for storing various types of data pertaining to the user, such as their medical history and treatment information. The natural language processing module captures the user's responses during treatment and transcribes them into written notes. This allows for easier understanding and sharing of the information within the system. The system and the method has potential applications for delivering mental health services through various channels, including interactive computer terminals and mobile applications. This could lead to increased accessibility, convenience, scalability, and potentially improved mental health outcomes for individuals and communities.

### CROSS-REFERENCE TO RELATED APPLICATIONS AND PRIORITY

The present application claims priority from PCT/IB2023/053984 filed on 19 Apr. 2023 further claimed Indian Patent Application 202211023699 dated 22 Apr. 2022, incorporated herein by a reference.

### TECHNICAL FIELD

The present subject matter described herein, in general, relates to a medical platform, more particularly to a medical self-help and/or need based platform for providing and receiving mental and behavioural health services, which includes potential applications for delivering mental and behavioural health services through various channels, including interactive computer terminals and mobile applications, with the aim of improving accessibility, convenience, scalability, and mental health outcomes for individuals and communities.

### BACKGROUND

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Mental or behavioural health is the most important aspect of one's life but unfortunately, it is also the most neglected field amongst all the medical branches since the beginning. Suicide due to depression is listed among the top causes of death according to the World Health Organization (WHO). Therefore, screening for depression and other mental health disorders by doctors and health service providers is widely recommended. A large variety of digital screening or monitoring tools for detecting behavioural health issues are available worldwide, which diagnose mental health not only by using assessment of natural language answers to a questionnaire, but also by assessment through calculating an Emotional Wellness Index (EWI) as disclosed in IN202111043914 enables achieving the objective.

In the modern world scenario, it has been observed that a significant percentage of all suicides committed by people are having no diagnosis of depression, on the other hand, a significant amount of people coping with stress may not have diagnosed themselves for depression. These two assertions appear entirely inconsistent with each other, screening or monitoring but, the reality is that not enough people are being screened. Possible reason for many people to be reluctant to seek help for the mental health issue, is due to stigma attached to mental illness, fear of discrimination, and the inconvenience of traditionally available mental and behavioural health servicing tools. This may result in delayed diagnosis and treatment, leading to poorer mental health outcomes for the individual.

Traditionally, mental health diagnosis has relied on clinical history taking by mental health professionals with option of adding either clinician administered or self-assessment scales to diagnose severity and depth of mental health disorder(s) followed by treatment either through a biomedical approach consisting of psychopharmacology or by addressing psychosocial aspect often lacking an integrative approach which ideally is required for best possible clinical outcome & comprehensive care.

For mental health service delivery, due to shortage of qualified mental health professionals & prohibitive costs, access to quality assessment & treatment of mental health disorders is unattainable for a significant portion of the population and the emergence of digital solutions as a promising avenue for mental health support has been hampered by operational issues and scalability challenges, which in turn limit their widespread impact and usage.

Thus, development of viable and scalable mental health technology platform with an integrated approach combining both biomedical & psychosocial services is an urgent need which will not only give holistic care to the masses on a single platform but also address stigma, improve psycho-social health, and facilitate & re-integration of patients into society with much better clinical outcomes & quality of life.

Besides covering up the digital mental wellness aspect, if we look at a brick & mortar model of mental health delivery system, when a patient visits a doctor with symptoms of a particular disease, the doctor examines the patient and determines the necessary diagnostic tests. The doctor would then write a referral note on a piece of paper, which the patient would have to physically carry with them to the diagnostic laboratory. This process can be time-consuming, cumbersome, and inconvenient for patients who may have to travel long distances to the laboratory, especially those living in rural areas. Moreover, once the diagnostic tests are completed, the patient would have to physically collect the test results and carry them back to the doctor. This process can be time-consuming and inconvenient for both the patient and the doctor. Similarly, if medication is required, the doctor would have to write a prescription on a piece of paper, which the patient would have to take to a physical pharmacy to purchase the medication. This process can also be time-consuming and inconvenient, especially if the patient is unwell and unable to travel. Overall, the conventional system of referring patients to diagnostic laboratories and pharmacies may be inefficient, time-consuming, and inconvenient for both doctors and patients. It may also result in a delay in diagnosis and treatment, leading to a potentially negative impact on the patient's health outcomes. Therefore, in mental health delivery system there is a need to develop an additional automated path labs & e pharmacies referral which may streamline the process of referring patients with mental health problems to diagnostic laboratories and pharmacies, respectively.

Adopting digital mental health technology can be an effective solution to this problem. Digital mental health platforms can offer integrated solutions that can streamline the referral process, provide online consultations, and enable secure communication between doctors, patients, and diagnostic laboratories and e-pharmacies. This can significantly reduce the burden on the patient and improve the overall healthcare experience for those seeking mental health care. Furthermore, such digital mental health platforms can also help reduce the stigma associated with mental illness by offering anonymity and privacy, which can encourage more people to seek help for mental health issues.

Conventionally the patient selects a professional caregiver based on the recommendation or reference given by friends and family. However, such recommendations or references are made without really understanding the exact need of the patient.

As known in the art, current mental and behavioural health servicing tools globally are limited to a clinic-based services, which acts as a clinical decision support service for the health care providers. These limited tools may not have capability to capture patient history and consider it before diagnosing or providing any recommendation. Further, a clinic based mental health servicing tool may have the capability to incorporate experience of patients and health care professionals limited to that clinic only.

Further there lacks a platform that enables “uberisation” of healthcare and counselling and pharmacotherapeutic treatment. The current platforms only focus on assisting the patient to connect with the doctor or healthcare professional. Further limitation of the current platforms is allowing access to the information across various platforms and applications. Further the current platforms also fail to actively interact with the patients to understand the type of the treatment that can be actively pursued by the patient.

For instance, a technology described in PCT Application Publication WO2021247792A1 relates to a systems and methods for mental health care delivery via artificial intelligence. This prior art encompasses a system and method for conducting mental health evaluations. It introduces an adaptive questionnaire based on AI-driven question selection, tailoring subsequent questions to the subject's responses in real-time. This approach is used to optimizes data collection efficiency by transforming responses into multiple subject intents, ultimately providing analysis results to healthcare practitioners.

Further, a technology described in US Application Publication US20210082563A1 relates to a methods for improving psychological therapy outcome. This prior art outlines a structured method for assessing therapist effectiveness in treating mental health disorders. It involves gathering patient and service variable data, assigning scores based on historical outcomes, and generating aggregate scores. Predictions of therapy outcomes are compared to observed outcomes after treatment, offering a systematic approach to therapist evaluation.

Furthermore, a technology described in US Application Publication US20210074406A1 relates to a pre-therapeutic and therapeutic digital medical device and method. This prior art focuses on pre-therapeutic processing to manage a patient's psychosocial well-being. It establishes an initial wellness score, employs a digital therapist interface, and teaches adaptive skills to address conditions like depression and adjustment disorders. Monitoring changes from the baseline score helps determine communication thresholds with healthcare providers, facilitating efficient communication between patients and their medical teams.

Additional purview of looking at the present psychiatric challenges is:

Psychiatric Problems—Present Global Challenges

Majority of the psychiatric disorders have a long gap between onset of symptoms, development of full-blown disorder and time to reach for treatment to mental health professionals leading to substantial decrease in quality of life, chronicity of illness & suboptimal clinical outcomes.

Preventive Aspect

If we can somehow identify individuals with the highest possibility of developing any emotional or psychiatric disorder followed by timely intervention, then we can either totally prevent the development of disorder by nipping the problem in the bud or delay the onset of disorder or reduce the severity significantly hence substantially reducing the burden of psychiatric disorders globally.

Curative Aspects

For those who are looking for cure, automated clarity of path followed by an integrative biomedical and psychosocial approach by utilizing services either through pharmacotherapy &/or counselling on a single digital platform can shift the paradigm from suboptimal to optimal treatment outcomes leading to better quality of life for patients besides decreasing morbidity and mortality due to psychiatric disorders significantly throughout the world.

Therefore, there is a long-felt need for a more efficient and a convenient system that can streamline the professional selection, referral process and improve the overall healthcare experience for users. Further, there is a need for a platform that enables storing of the medical history of the user and capturing the progress, treatment provided. Further there is a need for a platform that enables sharing of the information between the professionals like method of treatment and others without compromising the privacy of the user.

### SUMMARY

This summary is provided to introduce concepts related to a medical platform for delivering mental and behavioural health services through various channels, and the concepts are further described below in the detailed description. This summary is not intended to identify essential features of the claimed subject matter nor is it intended for use in determining or limiting the scope of the claimed subject matter.

In one embodiment of the present disclosure, a system for providing mental and behavioural health services is disclosed. The system may comprise a memory, a processor coupled to the memory, wherein the processor may be configured to execute program instructions stored in the memory, wherein the program instructions may correspond to a plurality of modules, wherein the plurality of modules may comprise a questionnaire module, an expressions capturing module, a decision module, a recommendation module, a professional module. The system may further comprise a network, one or more I/O interface, wherein the one or more I/O interface may be communicatively coupled to the system through the network. The processor may be configured to execute program instructions stored in the memory for receiving a user data, wherein the user data may comprise one or more responses provided by a user against a set of questionnaires. The processor may further be configured to execute program instructions stored in the memory for selecting a professional from a set of professionals based on the user data, wherein the set of professionals may comprise the professional module or a human professional. The processor may further be configured to execute program instructions stored in the memory for recommending the professional from the set of professionals to the user, wherein the professional may correspond to the professional module. Further, the processor may be configured to execute program instructions stored in the memory for providing mental and behavioural health services to the user based on the user data.

In another embodiment of the present disclosure, the processor may be configured to execute program instructions stored in the memory for interacting with the user to capture interaction data, wherein interacting may correspond to asking a set of randomly generated questions using the questionnaire module. The processor may be configured to execute program instructions stored in the memory for capturing a set of expressions of the user by using the expression capturing module. The processor may be configured to execute program instructions stored in the memory for integrating the interaction data and the set of expressions for an evaluation. The processor may be configured to execute program instructions stored in the memory for sharing a user detail to the human professional from the set of professionals, based on the evaluation, wherein the user detail may comprise the user data, interaction data and the set of expressions.

In one implementation of the present disclosure, a method for providing mental and behavioural health services is disclosed. The method may comprise steps of receiving a user data, wherein the user data may comprise one or more responses provided by a user against a set of questionnaires. The method may further comprise the step of selecting a professional from a set of professionals based on the user data, wherein the set of professionals may comprise a professional module or a human professional. The method may further comprise a step of recommending the professional from the set of professionals to the user, wherein the professional may correspond to the professional module. The method may further comprise a step of providing mental and behavioural health services to the user, by the professional module, based on the user data.

In another implementation of the present disclosure, the method may comprise a step of interacting with the user to capture interaction data, wherein interacting may correspond to asking a set of randomly generated questions using the questionnaire module. The method may comprise a step of capturing a set of expressions of the user by using the expression capturing module. The method may comprise a step of integrating the interaction data and the set of expressions for an evaluation. The method may comprise a step of sharing a user detail to the human professional from the set of professionals, based on the evaluation, wherein the user detail may comprise the user data, interaction data and the set of expressions.

The objective of the present disclosure is to provide holistic support from Prevention to Cure to the whole spectrum of Mental Wellness Solutions based on curated historical data.

Another objective of the present disclosure is to employ a hierarchical model configured to distinguish between the need for a psychologist or psychiatrist along with facilitating transition between professionals.

Yet another objective of the present disclosure is to capture how a pool of experts who are leaders in psychiatry and psychology provide treatment and further train the professional module using the historical data.

Yet another objective of the present disclosure is to leverage the worldwide presence of a doctor with global expertise to preserve and disseminate their recommendations through the healthcare system, enabling the provision of consistent and high-quality healthcare recommendations to individuals across multiple regions and countries, regardless of their physical location or proximity to the expert doctor.

Yet another objective of the present disclosure is to shift global paradigm of emotional and mental health services by empowering individuals which enable them to gain a deep understanding of their own mental health status and to equip people with the precise knowledge and insights necessary for making informed and deliberate decisions concerning their personal mental health care, hence democratising mental health care.

Yet another objective of the present disclosure is to render integrated biomedical and psychosocial approach for providing treatment to the community using holistic care model.

Yet another objective of the present disclosure is to deliver an aggregating solution like a global super app leading to holistic scalable service for mental health globally from prevention to cure.

### BRIEF DESCRIPTION OF DRAWINGS

The detailed description is described with reference to the accompanying Figures. In the Figures, the left-most digit(s) of a reference number identifies the Figure in which the reference number first appears. The same numbers are used throughout the drawings to refer like features and components.

FIG. 1 illustrates a network implementation (100) of a system (101) for providing mental and behavioural health services, in accordance with an embodiment of the present subject matter.

FIG. 2 illustrates a block diagram 200 for showing components of the system (101), in accordance with an embodiment of the present subject matter.

FIG. 3 illustrates a data flow representation method (300) for providing mental and behavioural health services, in accordance with an embodiment of the present subject matter.

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments. Following is an example which is illustrative only and invention accommodates any and every variation of the example provided below that shall serve the same purpose and is obvious to a person skilled in the art.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are described. The disclosed embodiments are merely exemplary of the disclosure, which may be embodied in various forms.

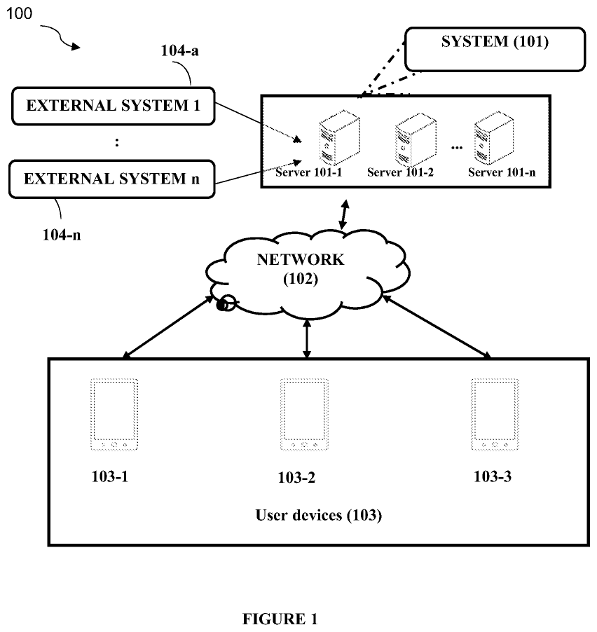

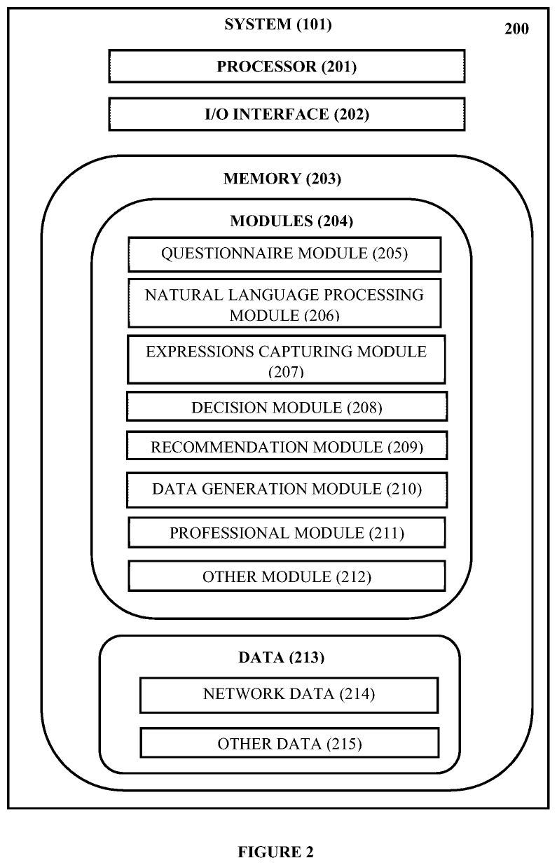

Referring to FIG. 1, a network implementation (100) for providing mental and behavioural health services is depicted, in accordance with an embodiment of the present subject matter. As shown in FIG. 1, the network implementation (100) for providing mental and behavioural health services may comprise a system (101), a network (102), one or more user devices (103), one or more servers (101-1, . . . , 101-n), and one or more External Systems (1-n). The system (101) may further comprise a memory (203, illustrated in FIG. 2), a processor (201) coupled to the memory (203), a one or more I/O interfaces (202) that may be communicatively coupled to the system (101) through the network (102).

The system (101) may be implemented using hardware, software, or a combination of both, including using where suitable, one or more computer programs, mobile applications or “apps” by deploying either on-premises over the corresponding computing terminals or virtually over cloud infrastructure. The system (101) may comprise various micro-services or groups of independent computer programs which can act independently in collaboration with other micro-services. The system (101) may also interact with a third-party or external computer systems. Internally, the system (101) may be the central processor of all requests for transactions by the various actors or users of the system. A critical attribute of the system (101) is that it is able to concurrently and instantly complete an online transaction by a system user in collaboration with other systems.

In another embodiment, the system (101) may be configured to use artificial intelligence (AI) or Machine Learning (ML) technology to provide mental health or behavioural services. artificial intelligence (AI) or Machine Learning (ML) technology may comprise technologies, for example but not limited to, Natural Language Processing (NLP) and/or Natural Language Understanding (NLU), ML model (e.g., Recurrent Neural Network (RNN), Convolutional Neural Network (CNN), deep neural network (DNN), and/or some other ML models), generative pre-trained transformer (GPT) family of language models, large language model (LLM), and the like This may involve incorporating AI or ML algorithms to analyse a user data and identify patterns or anomalies that may indicate the presence of mental health issues or behavioural disorders. The system (101) may also use artificial intelligence (AI) or Machine Learning (ML) technology to develop personalized treatment plans based on user data, previous treatment outcomes, and other relevant factors. By leveraging these technologies, the system (101) may potentially improve the accuracy and effectiveness of mental health and behavioural services and provide users with more targeted and personalized care.

In one embodiment, the system (101) may be configured to provide augmented human intelligence. In another embodiment, the system (101) may be configured to integrate with various applications such as, but not limited to, chatbot, personal assistant, voice assistant, Chat-GPT, simulation, metaverse, omniverse, virtual reality (VR), mixed reality (MR), augmented reality (AR), eXtended Reality (XR), and other futuristic technical development.

In one embodiment, the system (101) may be configured to be installed at one or more places, such as but not limited to, airport, railway station, bus stop, shopping mall market, office, coffee shops, restaurants, club, movie theatre, library, school, college, residential society, public park, public place and the like.

In one embodiment, the network (102) may provide the means for communication between the one or more user devices (103), the one or more servers (101-1, . . . , 101-n), the one or more External Systems (1-n), and the system (101). Further, the network (102) may enable the communication between the system (101) and the one or more I/O interfaces (202). In another embodiment, the network (102) may also enable the communication between the system (101) and other systems or devices, such as professional therapists or counsellors who may provide additional support to the user. In one embodiment, the network (102) may comprising any one of the following: a wireless network, a wired network, a telephone network (e.g., Analog, Digital, POTS, PSTN, ISDN, xDSL), a cellular communication network, a mobile telephone network (e.g., CDMA, GSM, NDAC, TDMA, E-TDMA, NAMPS, WCDMA, CDMA-2000, UMTS, 3G, 4G, 5G, 6G), a radio network, a television network or a combination thereof. The network (102) may be implemented as at least one of the different types of networks, such as intranet, local area network (LAN), wide area network (WAN), wireless local area network (WLAN) and/or wireless metropolitan area network (WMAN), the internet, an electronic positioning network, an X.25 network, an optical network (e.g., PON), a satellite network (e.g., VSAT), a packet-switched network, a circuit-switched network, a public network, a private network and the like. The network (102) may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), Institute of Electrical and Electronics Engineers (IEEE) standards, protocols, and variants such as IEEE 802.11 (“WiFi”), IEEE 802.16 (“WiMAX”), IEEE 802.20x (“Mobile-Fi”), and others, to communicate with one another. Further the network (102) may include a variety of network devices, including routers, bridges, servers, computing devices, storage devices, and the like. The network (102) may be configured to support short range communication such as a wireless personal area network (WPAN) communication, Bluetooth® data communication, infrared (IR) communication, near-field communication, electromagnetic induction (EMI) communication, passive or active RFID communication, micro-impulse radar (MIR), ultra-wide band (UWB) communication, automatic identification and data capture (AIDC) communication, and others.

In an exemplary embodiment of the present disclosure, one or more users may be configured to use the one or more user devices (103) or an interactive computer terminal in order to share the user data with the system (101). In one embodiment of the present disclosure, the user may be configured to use the user device (103) but not limited to laptop computer, a desktop computer, a notebook, a workstation, tablets, mobile devices, a portable computer, a personal digital assistant, a handheld device, and a workstation, a mainframe computer, a server, a network server, and the like. The user may be configured to share the user data with the system (101) by using but not limited to an embedded application in the user device (103) or by using the interactive computer terminal.

In another embodiment of the present disclosure, the user may be configured to use the interactive computer terminals but not limited to self-service kiosks, Automated Teller Machines (ATMs), digital booth, vending machines, Dispensing machine, point of sale (POS), Touchscreen terminal, interactive whiteboards, smartboards, Interactive displays, self-checkout machines, and other self-service machines and the like.

In accordance with the exemplary embodiment an AI based platform may be embedded in the system (101). It will be understood that the system (101) may be accessed by multiple users through one or more user devices 103-1, 103-2 . . . 103-N, collectively referred to as user devices (103) hereinafter, or applications residing on the user devices (103). Further the embedded application on the user devices (103) may be configured to use various hardware and sensors provided in the user devices (103) to capture the user's reactions while accessing the AI based platform on the system (101). The user devices (103) may be communicatively coupled to the system (101) through the network (102).

In one embodiment of the present disclosure, the one or more servers (101-1, . . . , 101-n) may be configured to enable communication between the one or more user devices (103) and the system (101). In one embodiment, the one or more servers (101-1, . . . , 101-n) may be configured to service requests from one or more user devices (103), the one or more External Systems (1-n), and the system (101). In another embodiment of the present disclosure, the one or more External Systems (1-n) may correspond to a one or more third party services which may be required to serve requests from the one or more user devices (103) and the system (101). In one embodiment, the one or more servers (101-1, . . . , 101-n) and the one or more External Systems (1-n) may be implemented using hardware, software, or a combination of both, including using where suitable, either on-premises over the corresponding computing terminals or virtually over cloud infrastructure.

Referring now to FIG. 2, a block diagram 200 comprising various components of the system (101) for providing mental or behavioural health services, is illustrated in accordance with an embodiment of the present subject matter. As shown, the system (101) may comprise a memory (203), a processor (201) coupled to the memory (201), and one or more input/output (I/O) interfaces (202). In one embodiment, the processor (201) may be implemented as microprocessors, microcomputers, microcontrollers, digital signal processors, central processing units, state machines, logic circuitries, and/or any devices that manipulate signals based on operational instructions. Among other capabilities, the processor (201) may be configured to fetch and execute computer-readable instructions stored in the memory (203).

The I/O interfaces (202) may comprise one or more input interfaces and one or more output interfaces. In one embodiment, the one or more input interfaces may comprise a variety of software and hardware interfaces, for example but not limited to, a keyboard, a mouse, a web interface, a graphical user interface, a touch screen interface, a camera, a microphone for taking voice input, sensors and the like. The one or more output interfaces may comprise a variety of software and hardware interfaces, for example but not limited to interactive whiteboards, smartboards, interactive display screen, speaker, tactile interface, and the like. In one embodiment, the I/O interfaces (202) may enable the system (101) to interact with a user directly or through the user devices (103). The users may interact with the system (101) by providing input through the input interfaces by using the user device (103). In one exemplary embodiment, this input may comprise responses to mental health questionnaires or other information about the user's mental state. The system (101) may process this input and generate output, which may be communicated to the user through the output interfaces. In the related embodiment, the output may comprise recommendations for treatment, reports on the user's mental state, or other information. Further, the I/O interface (202) may enable the system (101) to communicate with other computing devices, such as web servers and external data servers (not shown). The I/O interface (202) may facilitate multiple communications within a wide variety of networks and protocol types, including wired networks, for example, LAN, cable, etc., and wireless networks, such as WLAN, cellular, or satellite. The I/O interface (202) may comprise one or more ports for connecting a number of devices to one another devices or to another server.

In one embodiment, the memory (203) may comprise any computer-readable medium known in the art including, for example but not limited to, volatile memory, such as static random-access memory (SRAM) and dynamic random access memory (DRAM), and/or non-volatile memory, such as read only memory (ROM), erasable programmable ROM, flash memories, hard disks, optical disks, magnetic tapes, memory cards and cloud storage. In one embodiment, the memory (203) may comprise one or more modules (204) and a data (213).

In another embodiment, the programmed instructions or modules (204) may include routines, programs, objects, components, data structures, etc., which may perform particular tasks or implement particular abstract data types. In one implementation, the modules (204) may comprise a questionnaire module (205), a natural language processing module (206), an expression capturing module (207), an decision module (208), a recommendation module (209), a data generation module (210), a professional module (211) and other modules (212). In one embodiment, the other modules (212) may comprise programs or coded instructions that supplement applications and functions of the system (101). In one embodiment, the one or more modules (204) may be implemented using hardware, software, or a combination of both, including using where suitable, either on-premises over the corresponding computing terminals or virtually over cloud infrastructure.

In another embodiment, the data (213), amongst other things, serves as a repository for storing data processed, received, and generated by one or more of the modules (204). The data (213) may comprise a network data (214) and other data (215). The other data (215) may comprise data generated as a result of the execution of one or more modules in the other module (212).

In one embodiment of the present disclosure, the questionnaire module (205) may be configured to randomly generate a first set of questions from a plurality of questions stored in the memory (203). In another embodiment, the questionnaire module (205) may be configured to generate another set of questions from the plurality of questions stored in the memory, based on receiving input from at least, the user's response, the system (101), the one or more modules from the modules (204) and a combination thereof. The plurality of questions, stored in the memory (203) may comprise questions related to domestic life, situational/circumstantial, nature of the user, professional life, and clinical questions related to their mental health and behavioural concerns and the like.

In another embodiment of the present disclosure, the natural language processing module (206) may be configured to receive, process, and analyse natural language answers from the one or more users in response to the set of questions provided by the questionnaire module (205). In one implementation, the natural language answers may be in the form of text format, audio format or the like. In a related implementation, the natural language processing module (206) may comprise a Speech-to-text (STT) submodule, also known as voice recognition module, wherein the STT submodule may be configured to convert audio or voice into the textual format. In another related implementation, the natural language processing module (206) may comprise a text-to-speech (TTS) submodule, also known as voice synthesis module, wherein the TTS submodule may be configured to synthesis textual data into audio or voice. In another related implementation, the natural language processing module (206) may be configured to translate language from one language to another language. The translation of one language to another language may enable the system (101) to cater the users and/or health care professionals worldwide.

In another embodiment of the present disclosure, the expression capturing module (207) may be configured to capture the user's expressions, such as facial expressions, voice expression, gaze expressions, to help assess their mental state. In another embodiment, the decision module (208) may be configured to process the collected data and may use a decision-making algorithm to determine the user's mental health status. In a related embodiment, the decision module (208) may be configured to help in deciding further procedures of the mental and behavioural health services of the system (101). In an exemplary embodiment, the decision module (208) may be configured to decide whether the user needs a psychologist, or a psychiatrist based on a user data, wherein the user data may comprise user demographic profiles, current psychological/psychiatric status, medical status, medical history, current treatment information, and other physiological data of the user. In another embodiment, the recommendation module (209) may be configured to provide recommendations to the user based on the user data. In one implementation, the recommendations may comprise recommending appropriate health care professionals, to the user based on the user data. The health care professionals may correspond to either the professional module (211) or a human professional. In one embodiment of the present disclosure, the professional module (211) may comprise a set of instructions stored in the memory to deliver mental and behavioural health professional services. In a related embodiment, the professional module (211) may comprise an AI powered healthcare professional service module. In one embodiment of the present disclosure, the human professional may comprise a community of psychologists and psychiatrists or other professionals trained in mental health providing mental and behavioural health professional services worldwide.

In an exemplary embodiment, the recommendation module (209) may be configured to recommend the professional module (211) based mental and behavioural health services to the user, in case of mild condition of the user. In another exemplary embodiment, the recommendation module (209) may be configured to recommend the human professional based mental and behavioural health services, to the user, either in case of severe condition of the user or in case of referral from the professional module (211) or in case of referral from other human professionals.

The advantage of implementing the system (101) that it recommends the professional module (211) for mild conditions and directs users to human professionals for severe conditions is multi-fold. For user with mild conditions, this approach may ensure immediate access to treatment through the professional module (211), eliminating the need to wait for a doctor's appointment. This not only expedites the healing process but also reduces the burden on the human professionals, as user with mild conditions may receive prompt and effective care through the professional module (211), without the necessity for direct human consultation.

Simultaneously, this strategy may alleviate the stress on the human professionals, as they are primarily engaged in cases of severe conditions or those referred from the professional module (211). Users with severe conditions may receive the specialized care they require from the human professional. This balanced approach may optimize the healthcare resources, enhances the user experience, and promotes the efficient allocation of mental and behavioural health services, benefitting both users and professionals alike.

In another embodiment, the mental and behavioural health services may correspond to providing treatment, therapy or counselling based on the user data.

In another embodiment of the present disclosure, the data generation module (210) may be configured to generate data reports based on the data collected by at least one of the questionnaire module (205) or the expression capturing module (207), and a combination thereof. In a related embodiment, the data generation module (210) may be configured to generate data reports based on the processing and analysis performed by at least one of the natural language processing module (206), the expression capturing module (207), the decision module (208), the recommendation module (209) and a combination thereof. In accordance with the exemplary embodiments disclosed the data generation module (210) may be configured to consume the stored data pertaining to the user, which may include user's demographic data, treatment log, other diagnosis and medical history and treatment. The treatment logs and various replies provided by the user during the treatment may be captured by the natural language processing module (206) and transcribed into written notes, for ease of understanding and sharing within the system (101).

In another embodiment of the present disclosure, the system (101) may be configured to enable an evaluation of the mental and behavioural health services provided to the user, wherein the evaluation may correspond to the real-time evaluation of the mental and behavioural health services. In one embodiment, the evaluation of the mental and behavioural health services may correspond to check whether the treatment, therapy or counselling provided to the user is working or not. In one embodiment, the evaluation may comprise interaction with the user to capture interaction data, wherein interaction may correspond to asking a set of randomly generated questions using the questionnaire module (205), In another embodiment, the evaluation may comprise capturing a set of expressions of the user by using the expression capturing module (207). In another embodiment, the evaluation may comprise integrating of the interaction data and the set of expressions. In another embodiment of the present disclosure, the system (101) may be configured to securely share user details to other human healthcare professionals without compromising privacy of the user, wherein the user detail may comprise the user data, interaction data and the set of expressions. In one embodiment, the sharing of the user details to the other human healthcare professionals may be used to improve treatment and to provide better health services. Overall, the system (101) may use a combination of automated modules and human professionals to provide mental and behavioural health services to users.

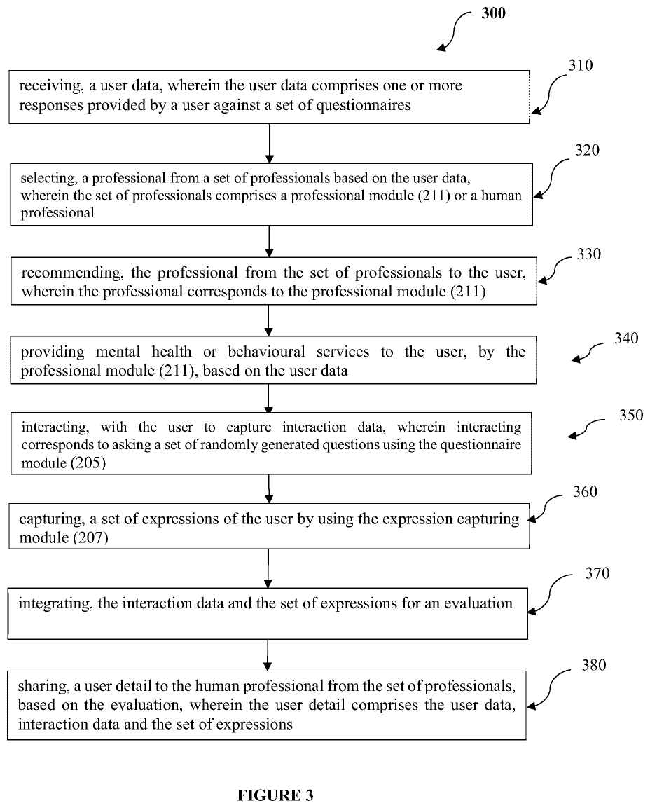

Referring to FIG. 3, data flow representation method (300) for providing mental and behavioural health services is illustrated, in accordance with an embodiment of the present subject matter. In a preferred embodiment, the method (300) may comprise the step (310) of receiving the user data, wherein the user data may comprise one or more responses provided by the user against the set of questionnaires. Further, the method (300) may comprise the step (320) of selecting the professional from the set of professionals based on the user data, wherein the set of professionals may comprise the professional module (211) or the human professional. Further, the step (330) may comprise, recommending the professional from the set of professionals to the user, wherein the professional may correspond to the professional module (211). Further, the step (340) may comprise, providing mental health or behavioural services to the user, by the professional module (211), based on the user data, wherein providing mental health or behavioural services may correspond to provide treatment or counselling based on the user data. Further, the next step (350) may comprise, interacting with the user to capture the interaction data, wherein interacting may correspond to asking the set of randomly generated questions using the questionnaire module (205). In the next step (360) may comprise, capturing a set of expressions of the user by using the expression capturing module (207), wherein the set of expression may comprise facial expression, voice expression or gaze expression. Further, the method (300) may comprise the step (370) of integrating the interaction data and the set of expressions for real-time evaluation. Finally in the step (380), sharing user details to the human professional from the set of professionals, based on the real-time evaluation, wherein the user detail comprises the user data, interaction data and the set of expressions.

In one embodiment, the system (101) and the method (300) may be configured to use artificial intelligence (AI) or Machine Learning (ML) technology to provide mental and behavioural health services. This may involve incorporating AI or ML algorithms to analyse the user data and identify patterns or anomalies that may indicate the presence of mental health issues or behavioural disorders.

In one exemplary embodiment, a user may use the user device (103) to access the system (101) via the I/O interface (202. The user may register themselves using the I/O interface (202) to access the system (101). In accordance with an exemplary embodiment of the present disclosure, a user may access the AI based platform embedded in the system (101) via the network (102). The AI based platform embedded in the system (101), retrieves the user data if it already exists on the AI based platform or may retrieve from another remote server. Once the data is retrieved, the recommendation module (209), may recommend various options for treatment and/or counselling. Further the recommendation module (209), may also recommend the professional module (211) or the human professional.

Some embodiments of the system (101) and the method (300) may be configured to create API's, which can be referred or utilized by other systems irrespective of their underlying technologies and implementation, for storing all those network data (214) in the event of usage of this AI based platform. The API may be shared with digital arm of one of, indoor acute inpatient psychiatry unit, rehabilitation centre, and a combination thereof.

Some embodiments of the system (101) and the method (300) may be configured to group one or more external systems (104) and logs of attempting answering to the first and the second set of questionnaires for all complex process-oriented subsystems which can be generated, with minimal computation costs.

Some embodiments of the system (101) may have capability to compare a predetermined set of values or data to be compared with the user input values or data. Some embodiments of the present system may provide notification to the plurality of external systems (104).

In accordance with an exemplary embodiment of the present disclosure, the mental and behavioural health services, both clinical and organizational, may be structured in a way that they may resemble an infinitely scalable, community-based virtual hospital. The mental and behavioural health services, disclosed herein, may be designed to be infinitely scalable in such a manner that they may expand and grow to meet the needs of a large population. This may be achieved through the use of technology and virtual platforms that may allow mental health professionals to provide services remotely and reach more people. The proposed system (101), providing mental and behavioural health services, may reach to the more people by implementing the service by using interactive computer terminals or by using mobile apps on the user devices (103) so that the services may be provided to the more people in a decentralized way and may be accessible to people wherever they are, rather than being limited to physical hospitals or clinics. This may allow mental health services to be delivered to individuals through a variety of channels such as interactive computer terminals which may be deployed at any public places such as railway/bus stations, airports, etc, and may also be made available on mobile applications. Therefore, the system (101) of present disclosure may be more accessible, convenient, scalable, and may potentially improve the mental health outcomes of individuals and communities.

In one exemplary embodiment of the present disclosure, the mobile application provided by the system (101) may be hereinafter referred as “Global digital mental health Super App” which may be integrated with any globally recognized cloud platform. This integration may aim to provide a global reach to the App while focusing on local requirements. The digital mental health super App is likely to offer mental health services or support through digital means, such as video conferencing, chatbots by asking questions to the user related to health. The integration of the “digital mental health app” with the globally recognized cloud platform may leverage the capabilities of the cloud platform, such as data analytics, security, and scalability. As a result, the App may be able to expand its reach to a global audience while meeting the local requirements of different regions or countries. The worldwide presence may assist the health service in preserving and disseminating recommendations provided by a single doctor who possesses global expertise. The same recommendation may be given by the system (101) to another individual or user or the doctor who may be in a nearby local hospital or the hospital in another region/country when it becomes applicable.

Further, in one exemplary embodiment of the present disclosure, the system (101) and method (300) for providing mental or behavioural health services may be designed to cater to different types of customers and business models such as D2C (Direct to Customer), B2C (Business to Consumer), B2B (Business to Business), B2G (Business to Government), B2I (Business to Institution) and A la carte model. Therefore, the single system may cater to all these different business models and allows customers to select specific services or products they require.

Further the system (101) and the method (300) may give automated indoor psychiatric hospitalisation referral which may help users to refer the treatment or doctor in the psychiatric hospitals for admission indoor treatment. The health service may take into account the user's medical history, current mental state, and risk of harm to themselves or others. If the system (101) determines that the user meets the criteria for psychiatric hospitalization, it may automatically generate a referral and send it to the appropriate hospital. This process may ensure that the user receives timely and appropriate care and may help to reduce the burden on emergency departments and mental health providers who may be overwhelmed with referrals.

Further, the system (101) and the method (300) may enable automated path labs & e pharmacies referral which may streamline the process of referring users to diagnostic laboratories and pharmacies, respectively. The automated path labs and e-pharmacies referral systems of the present disclosure may streamline the entire process of referring users to diagnostic laboratories and pharmacies. The doctor may use the software application to generate the referral note, which may then be sent electronically to the user's mobile phone or email. The user may then use the same system to book an appointment at a diagnostic lab of their choice and get the tests done. Once the test results are ready, they may be sent electronically to the doctor, who can access them through the same system. If medication is required, the doctor may generate an e-prescription, which may be sent electronically to an e-pharmacy. The user may use the same system to order the medication and have it delivered to their doorstep. Overall, automated path labs and e-pharmacies referral systems make the process of referring users to diagnostic labs and pharmacies more efficient and convenient for both doctors and users.

Further the system disclosed herein, may provide an interface for automated triaging by referring users to either a psychologist or psychiatrist, or both, after a screening process. This interface may offer both direct appointment-based services as well as digital screening and referral services. The automated triaging may help to streamline the process of accessing mental health services and ensure that users receive the appropriate level of care based on their needs. Further, the system may provide a single platform for specific user-based digital interaction amongst psychologists and psychiatrists to enhance clinical excellence. This platform may allow mental health professionals to collaborate and share expertise, ultimately leading to better user outcomes. By having a unified platform, mental health professionals may also be able to provide more comprehensive and coordinated care to users.

Nowadays, in the field of digital mental health, AI and machine learning may play a significant role. In the present disclosure, the proposed system may use AI & machine learning based upgradation in order to collect more data from users using digital mental health platforms. Further, AI and machine learning algorithms may be able to analyse this data to provide more specific clinical outcomes. This may help mental health professionals tailor treatments to individual users' needs, resulting in more effective care.

Further, in one exemplary embodiment of the present disclosure, there may be an aggregator model for mental health services. Further, with the capability to cater multi-lingual users across all age groups, this model may connect mental health professionals with users all over the world, making it easier for users to access care and for mental health professionals to offer their services from prevention to cure. This may be especially beneficial for underserved populations who may not have access to mental health services otherwise.

The Artificial Intelligence technology used in the system (101) and the method (300) may improve the collaboration and care management between older adults, caregivers, and care providers. With AI-augmented care management, there may be scalable solutions that provide more personalized care to older adults. By incorporating AI, there may be an opportunity to increase the agency of older adults in monitoring and managing their mental health with the help of their care team. Overall, the use of AI in care management can help to improve the quality of care, increase accessibility, and empower older adults to take a more active role in their own mental health care. In another embodiment, the system (101) and the method (300) may be used to provide a preventive mental and behavioural healthcare services to students, who generally are very prone to the distress. Further, Artificial Intelligence may help care providers make better clinical decisions, leading to enhanced care for users. The AI may help to analyse large amounts of clinical data and may extract important patterns and information. By providing this information to care providers, AI may help them to make more informed decisions about user care. In addition to improving decision-making, the use of AI may also lead to enhanced care by providing training and support for care providers. By using AI tools, care providers may learn about new treatment options, best practices, and emerging trends in their field, which may improve the quality of care they provide to users.

Overall, the integration of AI into clinical decision-making may help to improve user outcomes, enhance the skills and knowledge of care providers, and ultimately may lead to better healthcare for all.

The health service provider system (101) and the method (300) may analyse the data related to the behaviour and health of older adults and their caregivers. The data being analyzed may be a “multi-modal real-world data,” which means it is collected from various sources in the real world, such as electronic health records, wearable devices, and mobile apps. The specific type of data being analyzed may include but not limited to medication use, sleep patterns, physical activity levels, mood, geospatial location (i.e., where the person is located at different times), and social interactions. The AI may “infer” or make predictions based on this data, specifically including not only a person's clinical state (i.e., whether they have a mental health disorder or not), but also their lifestyle, including any potential risks or protective factors. By analyzing this data and making predictions, the healthcare providers may better understand a person's mental health status and may tailor their care accordingly. Here, the system (101) may outline the different stages of mental health care that may be supported by AI technology. The first stage is assessment, where the AI may help to recognize the symptoms and support diagnosis by analyzing the multi-modal real-world data, including medication use, sleep patterns, physical activity, mood, geospatial location, and social interactions. Further, the AI may make the risk assessment and prediction of mental health trajectories, where the AI may make predictions about a person's future mental health based on their current behaviour and lifestyle factors. This may help healthcare providers to develop personalized treatment plans that are tailored to each individual's needs. The second stage is interventions, where the AI may help healthcare providers to develop and deliver tailored interventions that are targeted at specific risk factors or protective factors identified in the assessment stage. Finally, the long-term monitoring, care management, and treatment response, where the AI may help healthcare providers to monitor a person's progress over time and adjust their treatment plan as needed based on ongoing analysis of their behaviour and lifestyle factors.

In accordance with the exemplary embodiment of the present disclosure, the additional AI based platform may recommend AI based counselling to the users having mild to very mild symptoms and pharmacotherapy to moderate to severe cases or a combination of both counselling & pharmacotherapy, as observed on the EWI (a self-help scale in mental health). The diagnosis offered by the Emotional Wellness Index (EWI) may represent a groundbreaking milestone in the field of mental health care. It proudly stands as the world's first self-help scale in mental health, marking a transformative global paradigm shift from reactive to proactive care in the field of emotional and mental health services. With the primary objective of the EWI being providing precision mental health insights, it may enable an individuals to gain a profound understanding of their own mental health status and make informed decisions about their mental health care, ultimately fostering a proactive stance towards emotional well-being. The AI based counselling/pharmacotherapy can be performed by the decision module (208) embedded within the platform, wherein the decision module (208) may be configured to provide specific remedial solutions based on algorithmic treatment methodology. The algorithmic treatment methodology may be further updated real-time to improve treatment, wherein the update may be based on analysis of historical data captured by the AI based platform and using machine learning and AI extract requisite information and create treatment model.

Further, another exemplary embodiment of the present disclosure may involve the treatment for the mental health through the groundbreaking “digital mental health App” application (a Global Super App). It may integrate both Bio-Medical and psychological approach, providing individuals with a comprehensive toolkit for managing their mental well-being with patients, counsellors/psychologists/doctors/psychiatrists as on a single platform for to & fro messaging besides counselling. This holistic approach may consider the intricate interplay between biological, psychological, and emotional factors, fostering a deeper and more effective treatment experience and remarkably improved clinical outcomes.

Within the aggregator platform for mental health service, the users may gain access to a 360 degree multitude of features that may encompass the entire spectrum of mental health support and management, from prevention to treatment on a single platform.

The disclosed system (100) may include following features:
1. Mental Health Support: The platform may be engineered to prevent, detect, and cure mental health issues, providing the users with a holistic approach to emotional well-being.
2. Counsellor/Psychologist discovery and consultation: The users may effortlessly find counsellors/psychologists, get counselled with them online, who securely access their health records and puts visit wise notes, facilitating seamless interactions with one or multiple such mental health professionals.
3. Health Reminders: Timely reminders for medication, therapy, and self-care routines may ensure that users stay on track with their mental health care plans.
4. Psychoeducation Repository: The system may house an extensive collection of mental health resources, enabling users to access valuable information and insights for their well-being.
5. Geographic and Dialect Agnosticism: Designed to transcend geographical and language barriers, the system (100) may be available and adaptable across various locations and languages, ensuring inclusivity and accessibility.
6. Electronic Health Records: The users may securely manage and access their health records online, streamlining the management of their mental health journey.

Therefore, the platform may establish a unified interface connecting patients, psychologists, and psychiatrists, offering integrated clinical services all within a single platform. It may cover the entire spectrum of mental health care, from prevention to cure, with just a click.

Further, the present disclosure's Community-Based Holistic Care Model not only addresses the challenges in mental health diagnosis, treatment, and digital solutions but also strives to redefine the landscape of mental health care by making it more accessible, personalized, and globally impactful.

The embodiments, examples and alternatives of the preceding paragraphs or the description and drawings, including any of their various aspects or respective individual features, may be taken independently or in any combination. Features described in connection with one embodiment are applicable to all embodiments unless such features are incompatible.

Although implementations for the system (101) and the method (300) for AI based platform using machine learning and AI technology have been described in language specific to structural features and/or methods, it is to be understood that the appended description is not necessarily limited to the specific features or methods described. Rather, the specific features and methods are disclosed as examples of implementations for real-time usage of the AI based platform.

1. A system for providing mental and behavioural health services, the system comprising:
a memory;
a processor coupled to the memory, wherein the processor is configured to execute program instructions, stored in the memory, wherein the program instructions are corresponding to a plurality of modules, wherein the plurality of modules comprise a questionnaire module, an expressions capturing module, a decision module, a recommendation module, a professional module;
a network;
one or more I/O interface, wherein the one or more I/O interface are communicatively coupled to the system through the network,
wherein the processor is configured to execute program instructions stored in the memory for:
receiving, by the system, a user data, wherein the user data comprises one or more responses provided by a user against a set of questionnaires,
selecting, by the system, a professional from a set of professionals based on the user data, wherein the set of professionals comprises the professional module or a human professional,
recommending, by the system, the professional from the set of professionals to the user, wherein the professional corresponds to the professional module, and
providing mental and behavioural health services to the user based on the user data.

2. The system as claimed in claim 1 further comprises, wherein the processor is configured to execute program instructions stored in the memory for:
interacting, by the system, with the user to capture interaction data, wherein interacting corresponds to asking a set of randomly generated questions using the questionnaire module,
capturing, by the system, a set of expressions of the user by using the expression capturing module,
integrating, by the system, the interaction data and the set of expressions for an evaluation,
sharing, by the system, a user detail to the human professional from the set of professionals, based on the evaluation, wherein the user detail comprises the user data, interaction data and the set of expressions.

3. The system as claimed in claim 1, wherein the system is configured to receive the user data captured by one or more user devices, wherein the user data comprises demographic profile, current medical status, medical history, current treatment information, and other physiological data.

4. The system as claimed in claim 1, wherein selecting the professional from the set of professionals is performed using the decision module.

5. The system as claimed in claim 1, wherein recommending the professional from the set of professionals is performed using the recommendation module, wherein the recommendation module is configured to recommend either the professional module or the human professional.

6. The system as claimed in claim 1, wherein the recommendation module is configured to recommend the professional module in case of a mild condition, wherein the recommendation module is configured to recommend the human professional in case of a severe condition.

7. The system as claimed in claim 1, wherein providing mental and behavioural health services comprise providing treatment and counselling based on the user data.

8. The system as claimed in claim 2, wherein the set of expressions of the user captured by using the expression capturing module comprises facial expressions, voice expressions, or gaze expressions.

9. The system as claimed in claim 2, wherein the evaluation is used to check whether the treatment or counselling by the professional module is working or not.

10. The system as claimed in claim 2, wherein the evaluation corresponds to a real-time evaluation while providing the mental and behavioural health services to the user.

11. The system as claimed in claim 1, wherein the system is configured to use artificial intelligence (AI) or Machine Learning (ML) technology to provide the mental and behavioural health services.

12. The system as claimed in claim 2, wherein the system is configured to facilitate secure sharing of the user data between the professionals and others without compromising the privacy of the user.

13. The system as claimed in claim 1, wherein the system is configured to provide the mental health, or the behavioural health services based on assessment through calculating an Emotional Wellness Index (EWI).

14. A method for providing mental and behavioural health services, the method comprising steps of:
receiving, a user data, wherein the user data comprises one or more responses provided by a user against a set of questionnaires,
selecting, a professional from a set of professionals based on the user data, wherein the set of professionals comprises a professional module or a human professional,
recommending, the professional from the set of professionals to the user, wherein the professional corresponds to the professional module, and
providing mental and behavioural health services to the user, by the professional module, based on the user data.

15. The method as claimed in claim 14, wherein the method further comprising:
interacting, with the user to capture interaction data, wherein interacting corresponds to asking a set of randomly generated questions using the questionnaire module,
capturing, a set of expressions of the user by using the expression capturing module,
integrating, the interaction data and the set of expressions for an evaluation,
sharing, a user detail to the human professional from the set of professionals, based on the evaluation, wherein the user detail comprises the user data, interaction data and the set of expressions.

16. The method as claimed in claim 14, wherein the recommending of the professional module is performed in case of a mild condition, wherein the recommending of the human professional is performed in case of a severe condition.

17. The method as claimed in claim 15, wherein the evaluation is used to check whether the treatment or counselling by the professional module is working or not.

18. The method as claimed in claim 15, wherein the evaluation corresponds to a real-time evaluation while providing the mental and behavioural health services to the user.

19. The method as claimed in claim 14, wherein the method is configured to use artificial intelligence (AI) or Machine Learning (ML) technology to provide the mental health or behavioural health services.

20. The method as claimed in claim 15, wherein the method is configured to facilitate secure sharing of the user data between the professionals and others without compromising the privacy of the user.
